(19) United States
(12) Patent Application Publication
(10) Pub. No.: US 2010/0160916 A1
(43) Pub. Date: Jun. 24, 2010

(54) CUTTTING DEVICE

(76) Inventors: Gursharan Singh Chana, West Midlands (GB); Christopher John Sloss, South Yorkshire (GB)

Correspondence Address: PATENTTM.US, P. O. BOX 82788, PORTLAND, OR, 97282-0788, US

(21) Appl. No.: 12/441,887

(22) PCT Filed: Sep. 25, 2007
(86) PCT No.: PCT/GB2007/003651

§ 371 Date: Mar. 10, 2010

(30) Foreign Application Priority Data
Sep. 27, 2006 (GB) 0618994.8
Apr. 26, 2007 (GB) 0708119.3

(51) Int. Cl. A61B 17/00

(52) U.S. Cl. 606/83

(57) ABSTRACT

A cutting device (11) comprises a cutting head (14) of substantially circular cross-section, the cutting head having a main axis (X), and a distal cutting edge (23). The cutting edge has a first cutter. The cutting head (14) has a cutting portion (21) and a second cutter extending inwardly of the cutting portion. The second cutter (26) comprises a plurality of spaced cutting members (32) arranged in an array around the main axis. Each cutting member has a substantially planar cutting surface (32A) extending substantially parallel to, and offset from, the main axis.

This invention relates to cutting devices. More particularly, but not exclusively, this invention relates to surgical cutting devices. Embodiments of the invention relate to femoral head cutters.

During hip replacement surgery, it is necessary to cut the patient's femoral head to the appropriate shape to allow the ball part of the replacement hip joint to be mounted thereon. Prior art cutting devices utilise one cutter to cut the head into a cylindrical configuration, and a further cutter to cut a chamfered region at the end of the head. The ball part of the replacement joint can then be mounted on the cut femoral head.

According to one aspect of this invention, there is provided a cutting device comprising: a cutting head of substantially circular cross-section, the cutting head having a main axis, a wall, and a distal cutting edge; the cutting edge having a first cutter; the wall having a cutting portion and a second cutter extending inwardly of the cutting portion; the second cutter comprising a plurality of spaced cutting members arranged in an array around the main axis; wherein each cutting member has a substantially planar cutting surface extending substantially parallel to, and offset from, the main axis.

The cutting portion may have a tapered face, and may be of a conical or frustoconical configuration. The cutting portion may have an inner face and the cutting members may be provided on the inner face of the cutting portion.

Each cutting surface is desirably arranged such that it extends parallel to, and is offset from, a respective plane in which the main axis lies.

Each cutting member may have a trailing surface, which may extend at an acute angle to the cutting surface. A cutting land may extend between the cutting and trailing surfaces of each cutting member.

Adjacent cutting members may have cutting and trailing surfaces facing each other. Thus, in the embodiments described herein, any line on any one of the cutting surfaces extending at right angles to the main axis of the cutting device extends parallel to any line on the adjacent facing trailing surface extending at right angle to the main axis.

The wall may comprise a cylindrical portion extending between the conical cutting portion and the cutting edge.

Each cutting member may be elongate, and may extend generally axially of the cutting head.

Apertures may be provided between adjacent cutting members. The apertures may be generally triangular. The apertures may have a base adjacent the cylindrical portion. The apertures may extend lengthwise of the cutting members, and may be substantially the same length as the cutting members.

The triangular apertures may narrow inwardly from the base along the length of the cutting members.

The cutting and trailing surfaces of adjacent cutting members may constitute two sides of the triangular apertures.

The first cutter may extend circumferentially relative to the second cutter. The second cutter may extend radially relative to the first cutter. The second cutter may extend inwardly relative to the first cutter.

The first and second cutters may be fixed relative to each other. The cutting device may comprise a surgical cutting device, which may comprise a bone cutter. The bone cutter may be configured for cutting a head of a bone, such as a femur. The cutting device may comprise a femoral head cutter.

The cutting head may be of a unitary construction, for example formed by casting or forging.

The cutting device may define a main axis. The cutting device may be rotatable about the main axis. The first cutter may be radially spaced from the main axis. The second cutter may be axially spaced from the first cutter.

The second cutter may extend radially inwardly relative to the first cutter. The second cutter may extend axially relative to the first cutter.

The first cutter may extend around the second cutter. The first cutter may be of a generally circular end profile.

The first cutter may comprise a plurality of cutting elements, which may be arranged in succession. The cutting elements may be arranged in a first substantially circular array. Preferably, the cutting elements are arranged around the circumference of the circular array.

The cutting elements may be spaced from one another. In one embodiment, the cutting elements may be substantially equispaced from one another. Each cutting element may have a first cutting surface. The first cutting surfaces of the cutting elements may be substantially coplanar relative to each other.

The first cutter may comprise a spacer member to space the first cutter axially from the second cutter. The spacer member may comprise a cylindrical portion

Each cutting element may face in an axial direction away from the second cutter.

In one embodiment, directing formations may extend from the cutting elements. The directing formations may be configured to direct from the cutting elements material cut by the cutting elements.

The directing formations may extend helically of the cutting head. Each directing formation may be generally triangular in configuration, and may have a broad region adjacent the cutting members and a narrow region spaced from the cutting members.

The directing formations may comprise channels defined in the cutting head.

The second cutting members may be arranged in a substantially circular array. Each cutting member may be provided with breaking formations to break off portions of the article to be cut. The breaking formations may comprise serrations or indentations, which may be arranged in a substantially helical array around the cutting portion. The breaking formations may comprise chip breakers, such as chip breaker serrations.

The breaking formations may extend along each cutting land.

The cutting head may comprise a tapered portion, such as a conical or frustoconical portion. The cutting members may be provided on the tapered portion. The tapered portion may have an inner face upon which the cutting members are provided.

The tapered portion of the cutting head may extend axially from the cylindrical portion of the cutting head.

The second cutting edge of each cutting member may extend axially relative to the first cutter and may extend inwardly relative to the first cutter.

In one embodiment, the first cutter may be a cylindrical cutter, configured to cut the first region of the article into a cylindrical form. The second cutter may be a chamfered cutter, configured to cut first second region of the article into a chamfered form, such as a frustoconical form.

The cutting device may include an elongate axially extending shaft upon which the first and second cutters are arranged. The shaft may define an axially extending guide bore to receive a guide member. The guide bore is desirably elongate, and may extend along the main axis.

The guide member may comprise an insertion member, which may be insertable into the guide bore of the shaft. The guide member may include a stop formation extending from the insertion member to restrict movement of the cutting device. The insertion member may be configured to be inserted into a hole in the article to be cut.

The insertion member may include a first insertion portion configured to be inserted into the bore in the shaft. The insertion member may also include a second insertion portion configured to be inserted into a hole in the article to be cut. The first insertion portion is preferably of a substantially cylindrical configuration to allow rotation of the cutting device about the main axis.

The stop formation may extend radially from the insertion member. The stop formation may be of a generally circular configuration.

The first insertion portion may extend in a first direction from the stop formation, and the second insertion portion may extend in the second direction from the stop formation. The first and second directions are preferably opposite to each other.

The cutting device may include an engagement face for engaging the stop formation and thereby restricting the axial movement of the cutting device relative to the guide member.

The elongate shaft may have a proximal end and a distal end. At the proximal end, the elongate shaft may comprise a securing formation for securing the cutting device to a suitable tool to effect rotation of the cutting device about its main axis, thereby allowing the cutting device to perform the cutting operation.

The first and second cutters may be provided at the distal end of the elongate shaft.

According to another aspect of this invention, there is provided a guide member for guiding a cutting device to cut an article, the guide member comprising an insertion member configured to be inserted into the cutting device and into a hole in the article to be cut, and further comprising a stop formation engageable with the cutting device and with the article to restrict movement of the cutting device relative to the guide member.

The guide member of this aspect of the invention may comprise features of the guide member described above.

According to another aspect of this invention, there is provided a cutting assembly comprising a cutting device as described above, and a guide member as described above, the guide member being configured to be inserted into the cutting device to guide the cutting device relative to the guide member.

According to another aspect of this invention, there is provided a method of producing a cutting device comprising: a) forming a cutting device a blank having a main axis and a cutting head blank having a substantially conical surface; b) providing a material removal tool having first, second and third orthogonal axes of movement; c) arranging the cutting device blank relative to the material removal tool such that the main axis of the cutting device blank and the first axis of the material removal tool do not cross each other; d) removing material from the cutting head blank to define an aperture therein; e) thereafter rotating the cutting device blank about the main axis and repeating steps (c) and (d) until a plurality of apertures are defined in the cutting head blank to provide a cutting head.

The step of removing material from the cutting head blank may comprise forming a cutting surface extending parallel to the main axis of the cutting device blank. During the step of forming the cutting surface, the first axis may be orthogonal to the conical surface.

During the step of removing the material from the cutting head blank, the material removal tool may remain oriented along the first axis, whereby substantially no angular movement of the material removal tool relative to the first axis occurs.

During the step of forming the cutting surface, the first axis may be offset from the main axis of the cutting device blank.

The step of arranging the cutting device blank relative to the material removal tool may comprise arranging the cutting head blank such that the first axis is substantially orthogonal to the conical surface and the first axis is offset from the main axis.

The step of removing material from the cutting head blank may comprise defining a generally triangular aperture therein.

According to another aspect of this invention, there is provided a method of using a cutting assembly as described above, comprising mounting the guide member on an article to be cut, arranging the cutting device on the guide member, such that the guide member is inserted into the cutting device, rotating the cutting device around the guide member, and moving the cutting device longitudinally towards the article along the guide member during rotation of the cutting device, so that the first cutter cuts a first region of the article, and moving the cutting device longitudinally further towards the article along the guide member during rotation of the cutting device, so that the second cutter cuts a second region of the article, said longitudinal movement continuing until the cutting device engages a stop formation on the guide member.

An embodiment of the invention will now be described by way of example only, with reference to the accompanying drawings, in which:

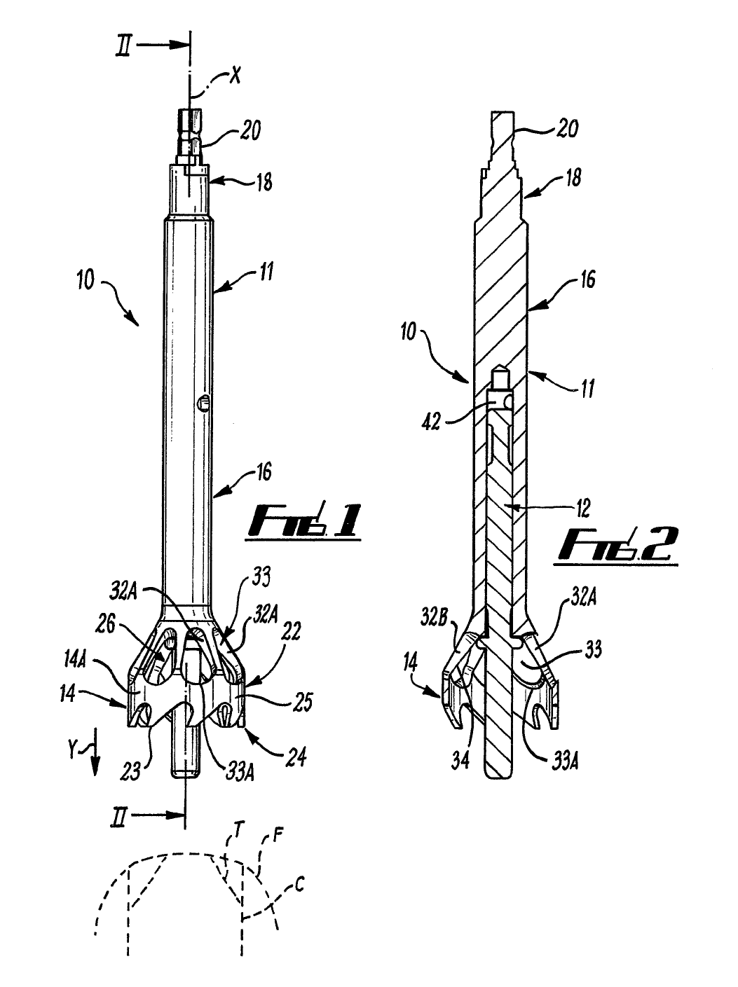

FIG. 1 is a side view of a first embodiment of a cutting assembly;

FIG. 2 is a sectional side view along the lines II-II in FIG. 1;

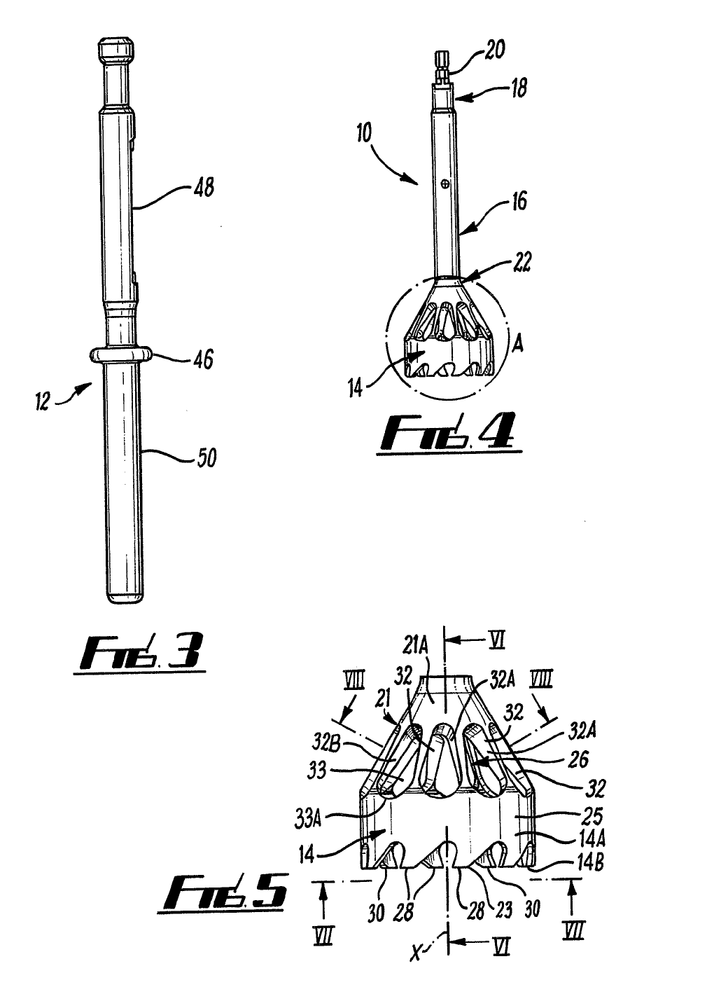

FIG. 3 is a side view of a guide member;

FIG. 4 is a side view of the cutting device shown in FIG. 1 without the guide member;

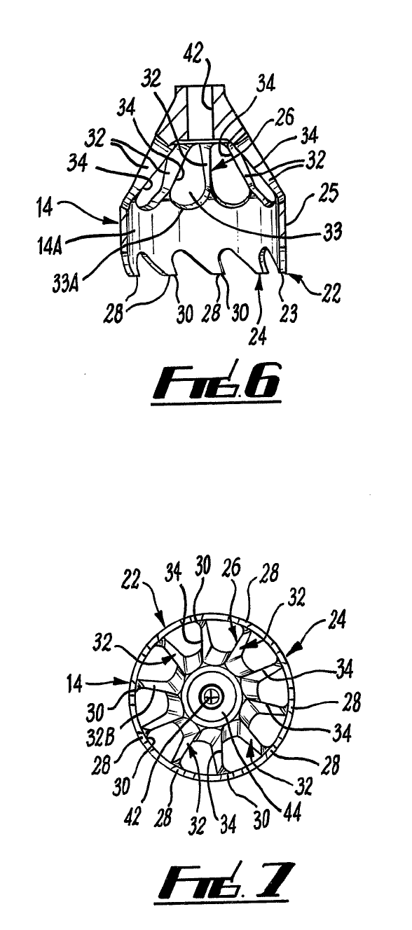

FIG. 5 is a close-up view of the region marked A in FIG. 4;

FIG. 6 is a sectional view along the lines 3 VI-VI in FIG. 5;

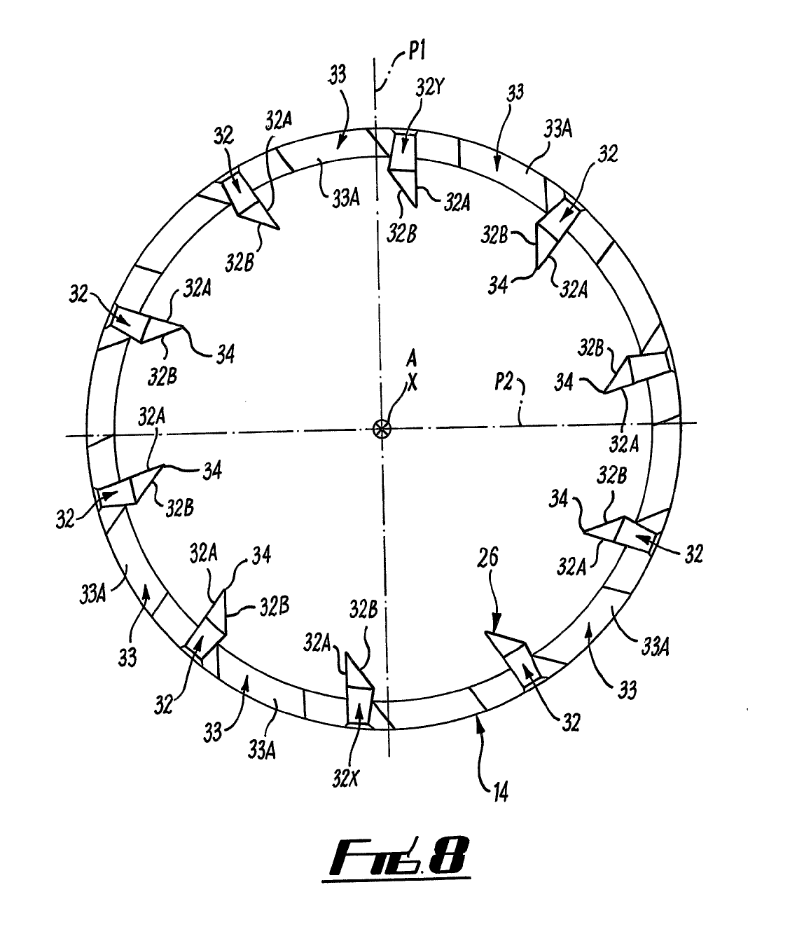

FIG. 7 is a view along the lines VII-VII in FIG. 5;

FIG. 8 is a cross-sectional view along the lines VIII-VIII in FIG. 5, where the view is taken at right angles to the surface of the cutting head;

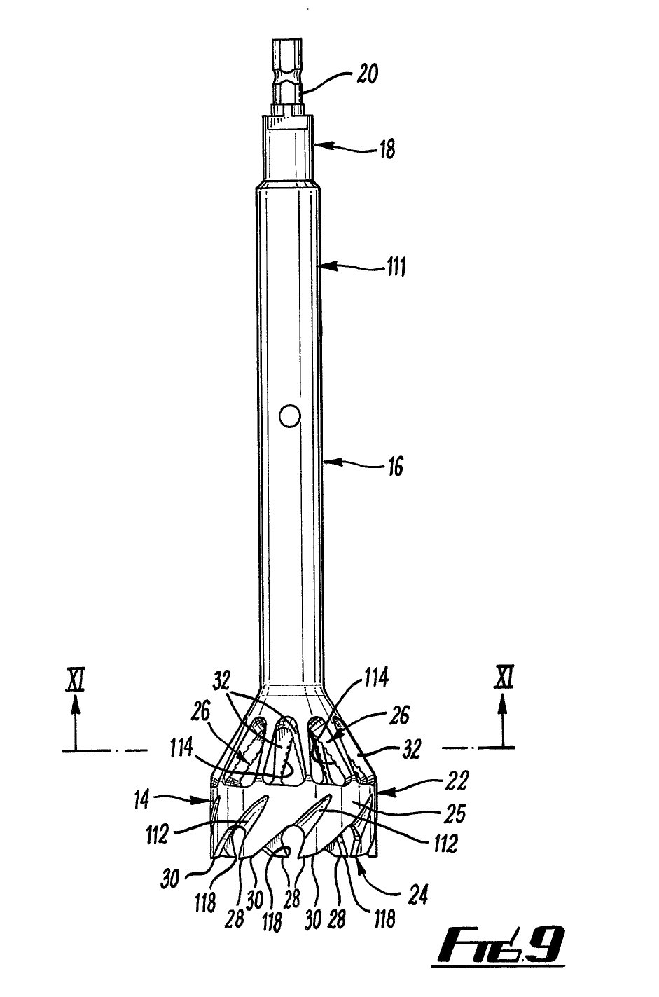

FIG. 9 is a side view of a second embodiment of a cutting device for use in a cutting assembly;

FIG. 10 is a perspective view of a cutting device shown in FIG. 9;

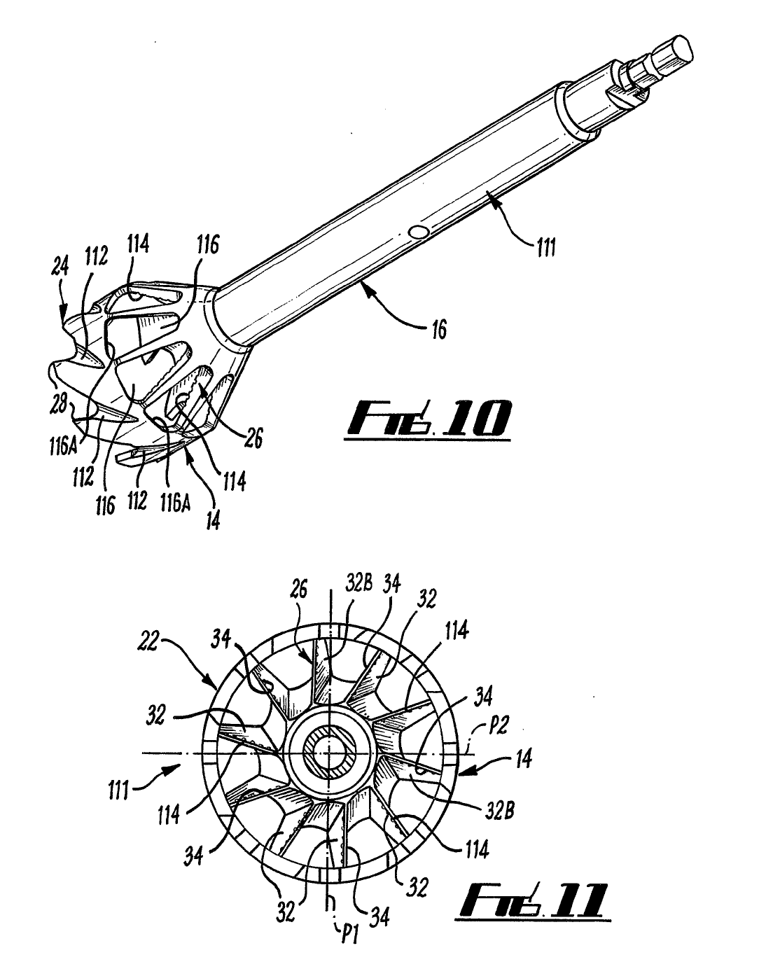

FIG. 11 is a view along the lines XI-XI in FIG. 9.

FIG. 12 is a diagrammatic side view of the a step in a method of producing a cutting device after formation of a cutting device blank;

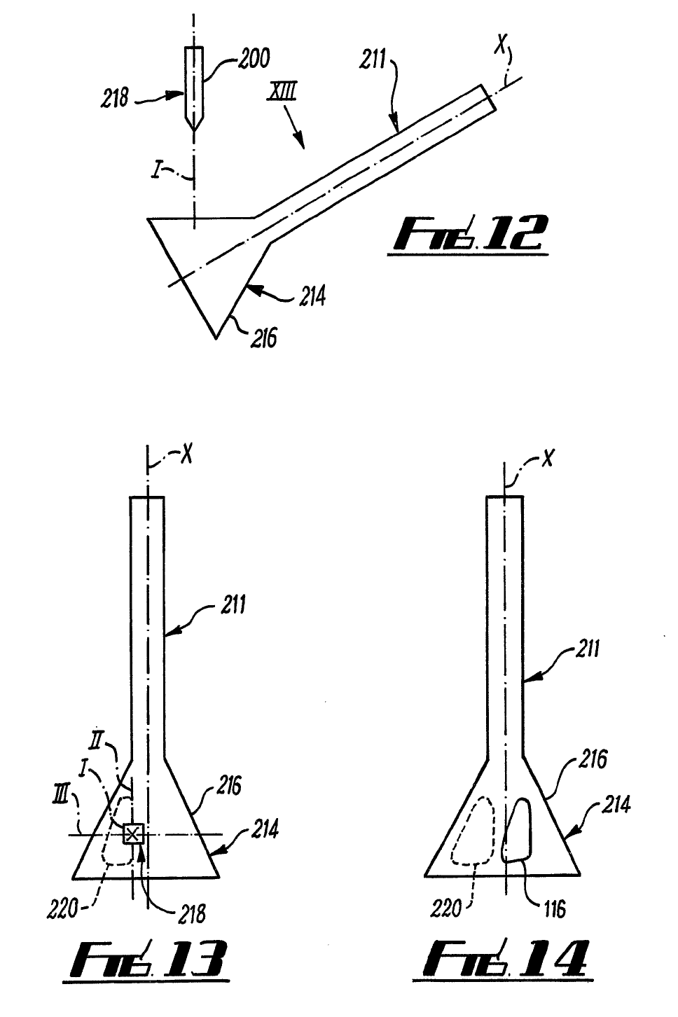

FIG. 13 is a view along the line XIII in FIG. 12; and

FIG. 14 is a view showing the method after rotation of the cutting device blank about its main axis.

FIGS. 1, 2 and 4 of the drawings show a cutting assembly 10 comprising a first embodiment of a cutting device, generally designated 11, and a guide member 12. The cutting device 11 is in the form of a femoral head cutter, suitable for cutting the head of a patient's femur during hip replacement surgery.

The head of the femur is shown diagrammatically in broken lines FIG. 1 of in the drawings, and designated F. A first embodiment of the cutting device 11 comprises a cutting head 14 on an elongate shaft 16. A main axis X extends through the elongate shaft 16 and the cutting head 14. The cutting device 11 is mounted on a power tool (not shown) to spin the cutting device 11 about the axis X, and is secured to the power tool by means of a securing formation 20, as described below.

The cutting head 14 and the elongate shaft 16 are, in the embodiment shown, formed as a one-piece unit, for example by casting or forging. Alternatively, the cutting edge 14 and the shaft 16 could be formed separately and fixedly connected together.

The elongate shaft 16 has a proximal end region 18 having a securing formation 20 to secure the cutting device 11 to power tool (not shown) to spin the cutting device 11 about its main axis to perform the cutting operation. The cutting device 11 also has a distal end region 22 at which the cutting head 14 is provided.

Referring to FIGS. 4 to 7, the cutting head 14 is shown which comprises a wall 14A, and a main axis X. The cutting head 14 includes a conical cutting portion 21 and a cylindrical portion 25, provided by the wall 14A.

A cutting edge 23 is provided at the distal end of the cylindrical portion 25. A first cutter 24 is provided on the cutting edge 23. A second cutter 26 is provided on the inner face of the conical cutting portion 21. The cylindrical portion 25 spaces the second cutter 26 from the first cutter 24.

The first cutter 24 comprises a plurality of cutting elements 28 arranged in a first circular array. Each of the cutting elements 28 is circumferentially equispaced from each adjacent cutting element 28.

The cutting elements 28 have respective first cutting edges 30 arranged substantially coplanar relative to each other and facing in an axial direction away from the second cutter 26.

The second cutter 26 comprises a plurality of equispaced cutting members 32 arranged in a second circular array. The first circular array of the cutting elements 28 is concentric with, the second circular array of the cutting members 32. Respective generally triangular windows 33 having a curved base 33A are defined between adjacent cutting members 32. The windows 33 allow for removal of pieces of bone cut by the cutting members 32.

As explained in more detail below with reference to FIG. 8, each cutting member 32 comprises a cutting surface 32A and a trailing surface 32B. The cutting surfaces 32A and the trailing surfaces 32B of adjacent cutting members 32 constitute two sides of the generally triangular windows 33.

Each cutting member 32 comprises an inwardly facing second land 34 that extends axially and radially inwardly relative to the first cutter 24. The cutting lands 34 are arranged at an oblique angle to the main axis X, in a frustoconical configuration. The cutting head 14 also includes an engagement face 40 for engaging a stop formation 46 on the guide member 12, as explained below.

Referring to FIG. 8, there is shown the sectional view of the cutting head 14 along the lines VIII-VIII in FIG. 5. Each of the cutting members 32 has a planar cutting surface 32A, and a planar trailing surface 32B, which extends at an acute angle to the cutting surface 32A.

As can be seen from FIG. 8, the cutting head 14 is of a circular cross-sectional profile having its centre on the central main axis X. FIG. 8 also shows planes P1 and P2 in which the main axis X lies. The planes P1 and P2 extend at orthogonally to the plane of the paper, and are only two examples of an infinite number of planes in which the main axis X lies.

The uppermost and lowermost cutting members 32, as shown in the drawing of FIG. 8, and which for ease of reference are designated 32X and 32Y, have cutting surfaces 32A which are offset from, and lie parallel with, the plane P1. Each of the other cutting members 32 also has a respective planar cutting surface 32A which also lies parallel to one of the planes in which the main axis X lies.

It is an advantage of the embodiments described in this specification that the cutting surfaces 32A are arranged in this manner. By having each cutting surface 32A lying parallel with, but offset from, a respective one of the planes in which the main axis A lies, the cutting members 32 can provide a much greater cutting efficiency than if the cutting surfaces 32A lie in one of the planes in which the main axis A lies.

Adjacent cutting members 32 have cutting surfaces 32A and trailing surfaces 32B which face one another. The adjacent facing cutting and trailing surfaces 32A and 32B are oriented as follows. A plane defined at right angles to a cutting surface 32A, and at right angles to the outer conical cutting surface 21A at the cutting surface 32A in question, extends at right angles not only to the cutting surface 32A in question, but also to the adjacent trailing surface 32B facing the cutting surface 32A. Thus, any line on any one of the cutting surfaces 32A extending at right angles to the main axis X of the cutting device extends parallel to any line on the adjacent facing trailing surface 32B extending at right angle to the main axis X.

Referring to FIGS. 2 and 3, FIG. 2 shows a cross-sectional view of the cutting device 11, and it can be seen that the shaft 16 defines an axial bore 42 extending along the main axis A-A (see FIG. 1). The guide member 12 is also shown in FIG. 2 in a cross-sectional view.

FIGS. 2 and 3 show the guide member 12, which can be slidably received in the axial bore 42 in the shaft 16. The guide member 12 guides the cutting device 11 during the axial and rotational movement of the cutting device 11.

The guide member 12 comprises an insertion member 44 and a stop formation 46 extending radially outwardly from the insertion member 44, about midway along the insertion member 44. The stop formation 46 has a circular profile.

The insertion member 44 comprises an elongate first insertion portion of 48 which is insertable into the bore 42 in the shaft 16 to guide the movement of the cutting device 11. The first insertion portion 48 extends from the stop formation 46 in a first direction.

The guide member 12 also includes an elongate second insertion portion 50 insertable into a hole drilled in the femoral head. In use, the second insertion portion 50 is inserted into the hole in the femoral head F until the stop formation 46 engages the bone.

The cutting device 11 is then arranged over the first insertion portion 48, now projecting from the femoral head F, and the first insertion portion 48 is received in the axial bore 42 in the shaft 16. The cutting device 11 has already been mounted on a suitable power tool to spin it about its main axis A-A.

As the cutting device 11 is spun about its main axis A-A, around the guide member 12, it is moved axially in the direction of the arrow Y in FIG. 1. The first cutter 24 engages the femoral head F, and subsequent movement of the cutting device 11 in the direction of the arrow Y causes the first cutter 24 to cut away the outer region of the femoral head F, thereby leaving a cut cylindrically shaped region, designated by the broken lines marked C in FIG. 1.

Further movement in the direction of the arrow Y causes the second cutter 26 to engage the now cylindrically shaped region C of the femoral head F. The obliquely angled second cutting edges 34 of the cutting members 32 cut the top of the cylindrically shaped region C into a chamfered region having a frustoconical shape, as shown by the broken lines marked T in FIG. 1.

The chamfered region T is continued to be cut as the cutting device 11 moves further in the direction of the arrow Y until the engagement face 44 on the cutting head 14 engages the stop formation of 46 in the guide member 12. This prevents further axial movement of the cutting device 11 relative to the guide member 12, thereby preventing any further cutting of the femoral head F.

Thus, the cutting assembly 10 is used to cut the femoral head of a patient into a cylindrical region having a frustoconical end, so that suitable hip replacement implants can be surgically mounted on the femur.

There is thus described a cutting assembly incorporating a cutting device that can be used during hip replacement surgery to cut the femoral head into the social implants to be used, and in which the embodiment described has the advantage that only one cutting device is required.

Various modifications can be made without departing from the scope of the invention. For example, the number of first and cutting members can be varied. Also, the angle of the cutting members can be altered dependent upon the shape of the implant to be mounted on the femoral head.

A second embodiment of the cutting device is shown in FIGS. 9, 10 and 11, and is generally designated 111. The cutting device 111 comprises many of the same features as the first embodiment of the cutting device shown in FIGS. 1, 2 and 4 to 8. these features have been designated with the same reference numerals as in FIGS. 1, 2 and 4 to 7.

The second embodiment of the cutting device 111 shown in FIGS. 8 to 11 differs from the cutting device 11 shown in FIGS. 1, 2 and 4 to 8 in that the cutting head 14 of the cutting device 111 includes directing formations in the form of triangular channel recesses 112. The triangular channel recesses 112 act to direct cut bone material from the femoral away from the cutting elements 24.

In addition, the cutting device 111 includes cutting members 32 having breaking formations in the form of chip breaker serrations 114 on the cutting edges 34. The chip breaker serrations 114 provide an improvement in the cutting of the femoral head by the cutting members 32 over the cutting device 111.

A further difference is that the cutting device 111 includes elongate triangular shaped windows 116 between adjacent cutting members 32. The triangular shaped windows 116 have a generally straight base edge 116A, and are larger than the windows 33 in the first embodiment of the cutting device 11. Thus, the cutting members 32 are longer in the second embodiment of the cutting device 111 than in the first embodiment of the cutting device 11.

In the second embodiment of the cutting device 111, the cutting elements 24 have a sharper cutting angle 118 than the cutting members 24 of the first embodiment of the cutting device 11. This provides the second embodiment of the cutting device 111 with an improvement over the first embodiment in the cutting of the femoral head by the cutting elements 24.

Referring to FIGS. 12 to 14, there are shown three of the steps in producing a cutting device 11′. The same method can also be used to form a cutting device 11.

Initially, a cutting device blank 211 is formed by any suitable known method, for example casting, such as investment casting. The cutting device blank 211 has a main axis X and a cutting head blank 214. The cutting head blank 214 has a conical outer surface 216.

The conical outer surface 216 is devoid of any of the triangular shaped windows 33 (or 116, if the second embodiment of the cutting device 111 is being produced). The triangular shaped windows 33 are to be formed by the method described below.

The cutting device blank 211 is arranged relative to a material removal tool 218, which can be any suitable material removal tool, such as a laser cutter. The material removal tool 218 is movable along first, second and third mutually perpendicular axes I, II and III.

The cutting device blank 211 is arranged as shown in the FIG. 12 such that the first axis I is perpendicular to the conical surface 216 of the cutting head blank 214 at the point where the first axis I crosses the conical surface 216. Also, as shown in FIG. 13, the first axis I is offset from the main axis X of the cutting head blank.

The first axis I remains offset from the main axis X during the removal of material from the cutting head blank 214.

Also during of the step of removal of material from the cutting head blank 214, the material removal tool 218 remains with the first axis I perpendicular to the main axis X, so that there is no angular movement of the material removal tool relative to the first axis I.

The material removal tool 218 then removes material from the cutting head blank 214 by cutting along a line represented by the broken line 220 in FIG. 13 to form the triangular window 33. The cutting device blank 211 is then rotated about the main axis X to a position shown in FIG. 14, in which a first of the generally triangular windows 116 has been formed by the method described above. The above steps are then repeated to form a second of the generally triangular windows 33, and then repeated again a desired number of times until all the generally triangular windows 33 have been formed.

In the embodiment shown, the cutting device 11 has ten of the generally triangular windows 33, which means that the cutting device blank 211 is rotated about its main axis X by 36° each time.

It will be appreciated by those skilled in the art that any other suitable number of windows 33 could be defined in the cutting head 14, depending upon the diameter or size of the cutting head 14, to enable it to achieve the desired cutting geometry. For example, where the cutting device 11 (or 111) is smaller, only eight of the windows 33 are formed in the cutting head blank. Thus, the cutting device blank 211 is rotated by angles of 45° each time one of the windows 33 is formed in the cutting head blank 214. It will also be appreciated by those skilled in the art that the description above, which applies to the first embodiment of the cutting device 11, also applies to the second embodiment of the cutting device 111.

1. A cutting device comprising: a cutting head of substantially circular cross-section, the cutting head having a main axis, and a distal cutting edge; the cutting edge having a first cutter; the cutting head having a cutting portion spaced from the cutting edge and a second cutter extending inwardly of the cutting portion; the second cutter comprising a plurality of spaced cutting members arranged in an array around the main axis; wherein each cutting member has a substantially planar cutting surface extending substantially parallel to, and offset from, the main axis.

2-52. (canceled)

53. A cutting device according to claim 1, wherein each cutting surface is arranged such that it extends parallel to, and is offset from, a respective plane in which the main axis lies, and wherein each cutting member has a trailing surface, extending at an acute angle to the cutting surface, and a cutting land extending between the cutting and trailing surfaces of each cutting member.

54. A cutting device according to claim 1, wherein the cutting head comprises a cylindrical portion extending between the conical cutting portion and the cutting edge, each cutting member being elongate, and extending generally axially of the cutting head, and triangular apertures are provided between adjacent cutting members, the apertures having a base adjacent the cylindrical portion, the triangular apertures extending lengthwise of the cutting members, and the apertures narrowing inwardly from the base along the length of the cutting members, and wherein the cutting and trailing surfaces of adjacent cutting members constitute two sides of the triangular apertures.

55. A cutting device according to claim 1, wherein the first cutter extends circumferentially around the second cutter and the second cutter extends radially relative to the first cutter, and wherein the second cutter extends inwardly relative to the first cutter.

56. A cutting device according to claim 1, wherein the first cutter is radially spaced from the main axis, and the second cutter is axially spaced from the first cutter and extends inwardly and axially relative to the first cutter.

57. A cutting device according to claim 1, wherein the cutting head comprises a spacer member to space the first cutter axially from the second cutter, the spacer member being of a generally cylindrical configuration.

58. A cutting device according to claim 1, wherein the first cutter comprises a plurality of spaced cutting elements arranged in succession circumferentially in a circular array, and wherein directing formations extend from the cutting elements the directing formations being configured to direct from the cutting elements material cut by the cutting elements.

59. A cutting device according to claim 58, wherein the directing formations extend helically from the cutting elements, each directing formation being generally triangular in configuration, and having a broad region adjacent the cutting members and a narrow region spaced from the cutting members.

60. A cutting device according to claim 1, wherein each cutting member is provided with breaking formations to break off portions of the article to be cut, the breaking formations extending along the second cutting edge, and comprise serrations or indentations extending in a substantially helical array around the inner face of the cutting portion.

61. A cutting device according to claim 1, including an elongate axially extending shaft upon which the first and second cutters are arranged, the shaft defining an axially extending guide bore to receive a guide member, wherein the guide bore is elongate and extends along the main axis, the guide member comprises an insertion member, which is configured to be inserted into the guide bore of the shaft, and the guide member includes a stop formation extending from the insertion member to restrict movement of the cutting device, the insertion member being configured to be inserted into a hole in the article to be cut.

62. A cutting device according to claim 61, wherein the insertion member includes a first insertion portion configured to be inserted into the bore in the shaft, and a second insertion portion configured to be inserted into a hole in the article to be cut, the first insertion portion being of a substantially cylindrical configuration to allow rotation of the cutting device about the main axis, wherein the first insertion portion extends in a first direction from the stop formation, and the second insertion portion extends in the second opposite direction from the stop formation, and wherein the cutting device includes an engagement face for engaging the stop formation and thereby restricting the axial movement of the cutting device relative to the guide member.

63. A cutting assembly comprising a cutting device as claimed in claim 1, and a guide member for guiding the cutting device to cut an article, the guide member comprising an insertion member configured to be inserted into the cutting device and into a hole in the article to be cut, and further comprising a stop formation engageable with the cutting device and with the article to restrict movement of the cutting device relative to the guide member the guide member being configured to be inserted into the cutting device to guide the cutting device relative to the guide member.

64. A cutting assembly according to claim 63, wherein the guide member comprises an insertion member, which is configured to be inserted into a guide bore in the cutting device, and the guide member further includes a stop formation extending from the insertion member to restrict movement of the cutting device, the insertion member being configured to be inserted into a hole in the article to be cut.

65. A cutting assembly according to claim 64, wherein the insertion member includes a first insertion portion configured to be inserted into the guide bore, and a second insertion portion configured to be inserted into a hole in the article to be cut.

66. A cutting assembly according to claim 65, wherein the first insertion portion is of a substantially cylindrical configuration to allow rotation of the cutting device about a main axis and wherein the stop formation is of a generally circular configuration and extends radially from the insertion member, the first insertion portion extending in a first direction from the stop formation, and the second insertion portion extending in a second opposite direction from the stop formation.

67. A method of producing a cutting device comprising: a) forming a cutting device a blank having a main axis and a cutting head blank having a substantially conical surface; b) providing a material removal tool having first, second and third orthogonal axes of movement; c) arranging the cutting device blank relative to the material removal tool such that the main axis of the cutting device blank and the first axis of the material removal tool do not cross each other; d) removing material from the cutting head blank to define an aperture therein; e) thereafter rotating the cutting device blank about the main axis and repeating steps (c) and (d) until a plurality of apertures are defined in the cutting head blank to provide a cutting head.

68. A method according to claim 67, wherein during the step of removing the material from the cutting head blank, the material removal tool remains oriented along the first axis, whereby substantially no angular movement of the material removal tool relative to the first axis occurs.

69. A method according to claim 68, wherein the step of removing material from the cutting head blank comprises arranging the first axis such that it is substantially perpendicular to the conical surface and offset from the main axis of the cutting device blank, and thereafter forming a cutting surface extending parallel to the main axis of the cutting device blank.

70. A method according to claim 68, wherein the step of arranging the cutting device blank relative to the material removal tool comprises arranging the cutting head blank such that the first axis is substantially perpendicular to the conical surface and the first axis is offset from the main axis.

71. A method according to claim 68, wherein the step of removing material from the cutting head blank comprises defining a generally triangular aperture therein.

72. A method of using a cutting assembly as described in claim 63, comprising mounting the guide member on an article to be cut, arranging the cutting device on the guide member, such that the guide member is inserted into the cutting device, rotating the cutting device around the guide member, and moving the cutting device longitudinally towards the article along the guide member during rotation of the cutting device, so that the first cutter cuts a first region of the article, and moving the cutting device longitudinally further towards the article along the guide member during rotation of the cutting device, so that the second cutter cuts a second region of the article, said longitudinal movement continuing until the cutting device engages a stop formation on the guide member.
